(19) United States
(12) Patent Application Publication
(10) Pub. No.: US 2025/0352525 A1
(43) Pub. Date: Nov. 20, 2025

(54) COMPOSITIONS AND METHODS OF TREATING TRAUMATIC BRAIN INJURY

(71) Applicants: United States Government As Represented By The Department Of Veterans Affairs, Washington, DC (US); University Of South Florida, Tampa, FL (US)

(72) Inventors: Subhra Mohapatra, Tampa, FL (US); Shyam S. Mohapatra, Tampa, FL (US); William Lawless, Tampa, FL (US); Wayne Guida, Tampa, FL (US); Ryan Green, Tampa, FL (US); Karthick Mayilsamy, Tampa, FL (US); Kristina Tosi, Tampa, FL (US)

(21) Appl. No.: 19/208,355

(22) Filed: May 14, 2025

Related U.S. Application Data
(60) Provisional application No. 63/647,415, filed on May 14, 2024.

(51) Int. Cl. A61K 31/44; A61K 31/404; C07D 213/74

(52) U.S. Cl. CPC A61K 31/44; A61K 31/404; C07D 213/74

(57) ABSTRACT

The disclosure relates to compositions including STING inhibitors. Methods of treating subjects with a traumatic brain injury by administering STING inhibitors are also included.

### CROSS REFERENCE TO RELATED APPLICATIONS

This application claims the benefit of the filing date of U.S. Provisional Application No. 63/647,415, filed on May 14, 2024. The content of this earlier filed application is hereby incorporated by reference in its entirety.

### STATEMENT REGARDING FEDERALLY SPONSORED RESEARCH

This invention was made with government support under grant numbers X005757, IK6BX004212, and IK6BX0060 awarded by the Department of Veterans Affairs. The government has certain rights in this invention.

### BACKGROUND

Traumatic brain injury (TBI) often results in long-term neurological damage due to inflammation from activated microglia. Current treatments focus on symptom relief and rehabilitation but fail to control the inflammatory and neurodegenerative processes directly. Thus, a need for new treatments exists.

### SUMMARY

Disclosed herein are methods of treating a subject with a traumatic brain injury, the methods comprising administering to the subject a composition comprising a therapeutically effective amount of H151, clonixeril, a ketone analogue of clonixeril, clonixin, a ketone analogue of clonixin, or a combination thereof, thereby treating the subject with the traumatic brain injury.

Disclosed herein are methods of reducing brain inflammation in a subject with a traumatic brain injury, the methods comprising administering to the subject a composition comprising a therapeutically effective amount of H151, clonixeril, a ketone analogue of clonixeril, clonixin, a ketone analogue of clonixin, or a combination thereof, thereby reducing brain inflammation the subject with the traumatic brain injury.

Disclosed herein are methods of reducing or inhibiting IBA1 or pSTING, the methods comprising administering to a subject a composition comprising a therapeutically effective amount of H151, clonixeril, a ketone analogue of clonixeril, clonixin, a ketone analogue of clonixin, or a combination thereof, thereby reducing or inhibiting IBA1 or pSTING.

Disclosed herein are methods of reducing expression of IRF3, IFNβ, CCL20, CCR6 or IL-1β in a subject, the methods comprising administering to the subject a composition comprising a therapeutically effective amount of H151, clonixeril, a ketone analogue of clonixeril, clonixin, a ketone analogue of clonixin, or a combination thereof, thereby reducing expression of IRF3, IFNβ, CCL20, CCR6 or IL-1β, in the subject.

Disclosed herein are methods of reducing expression of ionized calcium-binding adaptor molecule 1 (IBA1) in a subject, the methods comprising administering to the subject a composition comprising a therapeutically effective amount of H151, clonixeril, a ketone analogue of clonixeril, clonixin, a ketone analogue of clonixin, or a combination thereof, thereby reducing expression of IBA1 in the subject.

### BRIEF DESCRIPTION OF THE DRAWINGS

FIG. 1 shows that TBI can induce immune response protein expression in the hippocampus.

FIG. 2 shows how TBI can be affected by STING.

FIG. 3 shows the structures of H-151, Clonixeril, and clonixin butyl ketone.

FIGS. 4A-B show that clonixeril (CXL) inhibits IBA1 and pSTING in IMG cells. FIG. 4A shows immunohistochemical staining of IBA1 and pSTING in LPS stimulated IMG cells treated with CXL. FIG. 4B shows integrated density of IBA1 and pSTING in LPS stimulated IMG cells treated with CXL. Error bars represent mean ±standard deviation (SD). Statistical significance was determined using one-way ANOVA; n=3 images/group; ***p<0.001, #p<0.05.

FIGS. 5A-B show that H151, ketone analog of clonixin (e.g., clonixin butyl ketone), and clonixeril inhibit IBA1 in BV2 cells. FIG. 5A shows immunohistochemical staining of IBA1 in PolyIC stimulated BV2 cells treated with STING inhibitory compounds. FIG. 5B show the integrated density of IBA1 in polyIC stimulated BV2 cells treated with STING inhibitory compounds. Error bars represent mean ±standard deviation (SD). Statistical significance was determined using one-way ANOVA; n=3 images/group; *p<0.05, **p<0.01.

FIGS. 6A-B show that Clonixin Butyl Ketone (referred to as “Ketone”) reduces CCL20, IL-6 and Irf-3. FIG. 6A shows qPCR for CCL-20, IL-6 and Irf-3 in poly-IC-stimulated BV2 cells treated with Clonixin Butyl Ketone. FIG. 6B shows qPCR for CCL-20, IL-6 and Irf-3 in poly-IC-stimulated BV2 cells treated with H151.

FIGS. 7A-B show that Clonixin Butyl Ketone inhibits p-STING. FIG. 7A shows Western blot for pSTING in poly-IC-stimulated HT22 hippocampal cells pretreated with Clonixin Butyl Ketone. FIG. 7B shows relative expression of pSTING in polyIC stimulated HT22 cells treated with Clonixin Butyl Ketone (10 μg/ml).

FIGS. 8A-B show the inhibition of pSTING in HT22 hippocampal neuronal cells. FIG. 8A shows the immunohistochemical staining of pSTING in polyIC-stimulated HT22 cells pretreated with STING inhibitory compounds. FIG. 8B shows the integrated density of pSTING expression in polyIC-stimulated HT22 mouse hippocampal cells treated with STING inhibitory compounds. Error bars represent mean ±standard deviation (SD). Clonixin Butyl Ketone is referred to as “Ketone”. Statistical significance was determined using one-way ANOVA; n=3 images/group; ***p<0.001.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for a traumatic brain injury, such as, for example, prior to the administering step.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be traumatic brain injury.

“Inhibit,” “inhibiting” and “inhibition” also mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

The terms “reducing,” “inhibiting” and “ameliorating,” as used herein, when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

A ketone is an organic compound with the structure R—C(═O)—R′, where R and R′ can be a variety of carbon-containing substituents. Ketones contain a carbonyl group —C(═O)—(a carbon-oxygen double bond C═O).

The terms “analog” or “analogue” as used herein, refer to a structure which is similar to another structure, except that one atom or group is replaced by another (similar-behaving) atom or group.

The terms “ketone analog” or “ketone analogue” as used herein, refers to a compound that mimics the properties of ketones, which are organic compounds characterized by a carbonyl group (—C═O) bonded to two carbon atoms. The term “ketone analogue of clonixeril” refers to a chemical derivative of clonixeril. The term “ketone analogue of clonixin” refers to a chemical derivative of clonixin. In some aspects, the ketone analogue of clonixin can also be referred to as “Clonixin Ketone Analog”. In some aspects, the ketone analogue of clonixin can be clonixin butyl ketone.

In some aspects, an examples of a ketone analogue of clonixin can be {2-[(3-chloro-2-methylphenyl)amino]pyridin-3-yl}-1-pentanone wherein the glycerol functional group of clonixeril is replaced at the ester group by a ketone with four repeating carbon atoms. {2-[(3-chloro-2-methylphenyl)amino]pyridin-3-yl}-1-pentanone

(C₁₇H₁₉CIN₂O, mw: 302.799 g/mol)

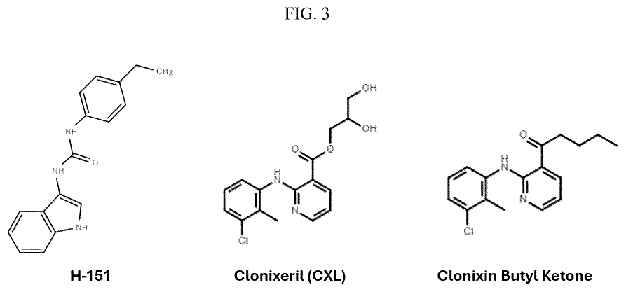

Clonixin Butyl Ketone can have the following chemical structure (see, FIG. 3):

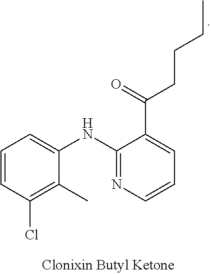 [SMILES: CCCCC(=O)c1cccnc1Nc1cccc(Cl)c1C]

Concussions, also referred to as traumatic brain injuries (TBI), happen when the head is injured or when there's a strong jolt to the body. They are a major cause of death and disability in the United States, including among Veterans. Even a small accident can lead to a serious head injury. Secondary damage following TBI is caused by complications such as neurovascular inflammation and excitotoxicity. Prevention of the resulting damage from these states is a major focus of TBI research. People with TBI experience long-term damage in the brain that leads to inflammation, cerebral neurodegeneration, retinal degeneration, microgliosis, brain cell damage, problems with vision, and changes in certain immune cells. Between 2000 and 2019, there were 414,000 reported cases of TBI among active-duty service members and Veterans. TBI and the problems that come with it are a major cause of disability in Veterans. Some of these issues include but are not limited to: disruption of cognitive function (challenges with memory and cognitive abilities), motor impairment (difficulty with movement and physical coordination), loss of sight and hearing, and higher risk of suicide. Eighty-five percent of veterans with severe TBI are unemployed, and 2.3 time more likely to develop dementia following TBI.

A protein called C—C-chemokine 20 (CCL20) and its partner receptor (CCR6) play an important role in causing this damage, promoting degenerative states, and worsening brain degeneration. Another protein called STING (stimulator of interferon genes), found in the endoplasmic reticulum (a part of cells), helps the immune system function, performing a principal function within the innate immune system. However, when STING is active, it increases CCL20 and CCR6 levels, which makes the inflammation in the brain worse after a TBI. Many of the above disabilities are directly linked to chemokine activity in TBI from CCL20 and CCR6, which in turn is partially regulated by the STING pathway. Chronic STING activation has been shown to play a role in autoimmune disease manifestation, studies have also suggested that enhanced STING signaling is important for anticancer immune response. Hence, some small molecule inhibitors at nanomolar concentrations have been identified to inhibit STING activity including H-151 that is believed to prevent STING dimerization and clonixeril, glycerol ester of a known NSAID clonixin. It's been well established that NSAIDs come with adverse effects affecting the gastrointestinal, renal, cardiovascular, and hematological systems. Hence, highly potent inhibitors effective in pico to low nano molar concentrations remain a major unmet need.

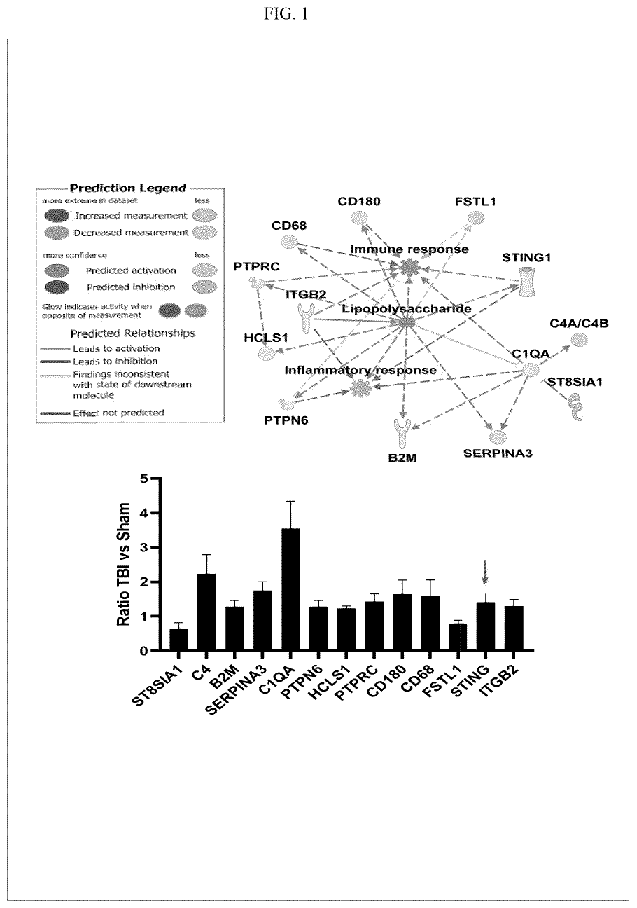

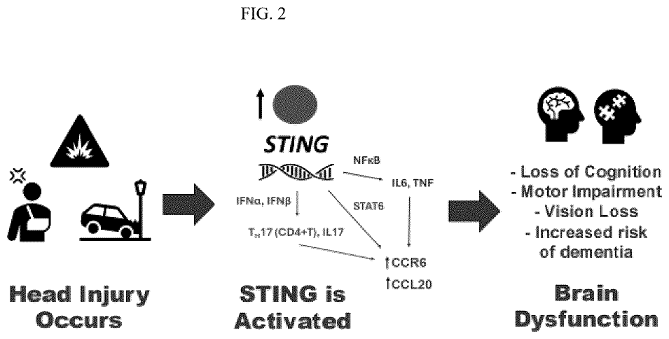

Based on an IPA analysis of genes involved in TBI relating to CCL20, which identified STING as the controller of inflammation and innate immunity upstream of CCL20 and CCR6 axis (FIG. 1), the goal of these investigations is to identify highly potent inhibitors effective in pico to low nano molar concentrations to treat traumatic brain injury as described herein is to help Veterans recover better from brain injuries, improve their quality of life, and lower long-term costs from disability (FIG. 2).

Described herein are compositions and methods of specifically inhibiting the STING pathway, an important driver of inflammation post-TBI. By targeting this pathway, the compositions and methods disclosed herein can reduce or inhibit microglial activation and inflammation, thus, providing a therapeutic strategy to protect neural tissue and improve recovery, distinguishing it from broader anti-inflammatory treatments.

### Compositions

Disclosed herein are compositions comprising: H151, clonixeril, a ketone analogue of clonixeril, clonixin, a ketone analogue of clonixin, or a STING inhibitor. In some aspects, the compositions can comprise clonixin butyl ketone. In some aspects, the compositions can further comprise a pharmaceutical acceptable carrier.

In some aspects, the STING inhibitor can be H-151. H-151 is a potent, irreversible, and selective small-molecule inhibitor of STING. H-151 exerts its inhibitory action by covalently binding to STING at the transmembrane cysteine residue at position 91. H-151 blocks STING palmitoylation and clustering, two important steps for STING signaling. H-151 can have the chemical structure (see FIG. 3):

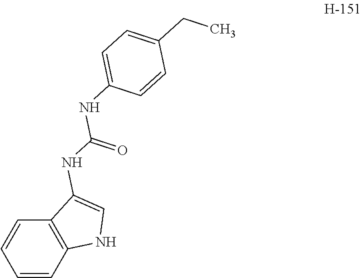 [SMILES: CCc1ccc(NC(=O)Nc2c[nH]c3ccccc23)cc1]

In some aspects, the STING inhibitor can be Clonixeril. Clonixeril can have the following structure (see, FIG. 3):

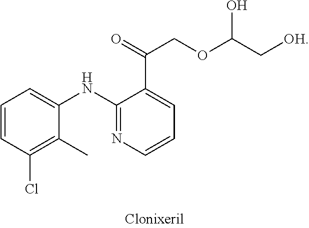 [SMILES: Cc1c(Cl)cccc1Nc1ncccc1C(=O)COC(O)CO]

In some aspects, the STING inhibitor can be one or more ketone analogues or derivatives of Clonixeril (CXL) or clonixin. In some aspects, the STING can be one or more ketone analogues of clonixin or clonixeril. In some aspects, the STING inhibitor can be clonixin butyl ketone. In some aspects, the STING inhibitor can be C-170, C-171, or H-151.

C-170 is a covalent small-molecule inhibitor of STING. C-170 efficiently inhibits both hsSTING and mmSTING through the same covalent modification with C-171. C-170 can have the formula C₁₅H₁₆N₂O₄.

C-171 is a covalent small-molecule inhibitor of STING. C-171 efficiently inhibits both hsSTING and mmSTING through covalently target the predicted transmembrane cysteine residue 91 and thereby block the activation-induced palmitoylation of STING. C-171 can also be referred to as N-(4-hexylphenyl)-5-nitro-2-furancarboxamide.

In some aspects, the STING inhibitor can be a nitro-fatty acid derivative, C-176, C-178, BPK-21/25, astin C, compound 18, SN-011, palbociclib, compound 30, 6,5-heterocyclic derivatives, or SP23. In some aspects, the STING inhibitor can inhibit STING signaling by covalently modifying Cys91 and preventing its palmitoylation. In some aspects, the STING inhibitor can inhibit STING signaling by covalently modifying Cys88 and preventing its palmitoylation. In some aspects, the STING inhibitor can inhibit STING signaling by preventing its palmitoylation. In some aspects, the STING inhibitor can inhibit STING signaling by binding to the cyclic dinucleotide binding site (CDN).

In some aspects, the ketone analogue of clonixin can be Clonixin Butyl Ketone.

In some aspects, the ketone analogue of clonixin can be one or more of the compounds or structures listed below:

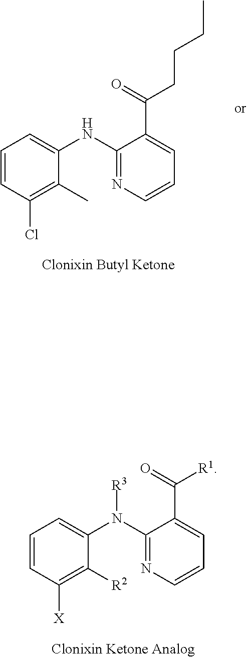

To synthesize clonixeril, the carboxylic acid precursor, clonixin (1.01), can first be produced by combining 2-chloronicotinic acid with 3-chloro-2-methylaniline with a catalytic amount of para-toluenesulfonic acid in water. After being heated to reflux overnight, the solution is cooled slightly before KOH is added to dissolve any product that may precipitate out. After filtration, concentrated sulfuric acid is added dropwise to the reaction and the resulting precipitate is filtered and recrystallized with methanol to yield the product as a white solid.

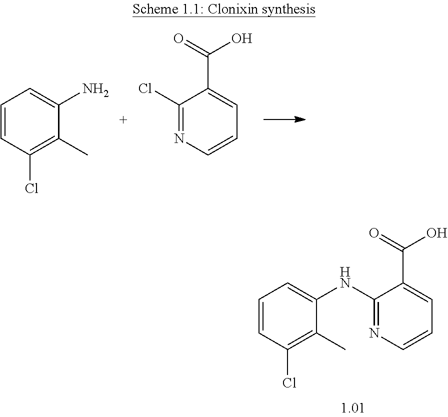

Reagents and conditions: pTsOH, H₂O, Δ.

The glycerol tail can be appended to the carboxylic acid moiety via EDC coupling procedures (Scheme 1.1). As glycerol contains two symmetrical primary alcohols and one secondary alcohol, to ensure that the primary alcohol is coupled to the acid, a protecting group is needed. Solketal, also known as DL-1,2-isopropylideneglycerol, is a racemic mixture of glycerol with an acetonide protecting group. Utilizing solketal ensures that the esterification occurs at a primary alcohol; deprotection of the acetonide is accomplished with the addition of aqueous acid. As this protecting group is acid labile, carbodiimide coupling procedures (i.e., Steglich esterification) are performed.

The Steglich esterification is performed using N,N′-dicyclohexylcarbodiimide, or DCC. However, the byproduct to this reaction, N,N′-dicyclohexylurea, is difficult to separate from instant product, so 1-ethyl-3-[3-dimethylaminopropyl]carbodiimide hydrochloride (EDC·HCl) is used.

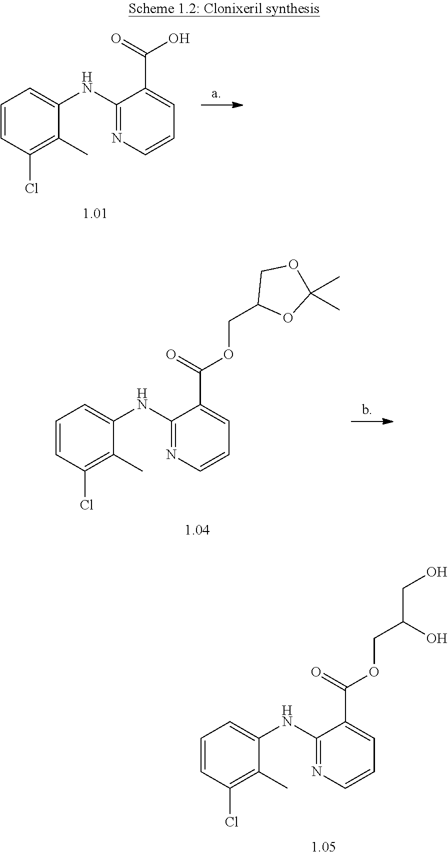 [SMILES: Cc1c(Cl)cccc1Nc1ncccc1C(=O)O ; Cc1c(Cl)cccc1Nc1ncccc1C(=O)OCC1COC(C)(C)O1 ; Cc1c(Cl)cccc1Nc1ncccc1C(=O)OCC(O)CO]

Reagents and conditions: a) EDC·HCl, DMAP, DCM, 0° C.-→rt; b) 6 M HCl, rt. To synthesize clonixeril (Scheme 1.2), 1.01 was dissolved in DCM at 0° C. before the addition of EDC·HCl. Excess solketal and catalytic DMAP is added to the reaction before the solution is allowed to warm to room temperature and stir overnight. Upon reaction completion, the crude reaction mixture is extracted with 2 M HCl and the crude organic material 1.04 is dissolved in THF before 6 M HCl is added. The acidified solution is allowed to stir at room temperature for 30 minutes before the organic material is re-extracted with the addition of brine and ethyl acetate. The organic layer is isolated, dried, and purified via CC to yield 1.05 as an oil.

To synthesize clonixin butyl ketone, a mixture of 3-chloro-2-methylaniline (1.67 ml, 14.0 mmol), 2-chloronicotinic acid (2.00 g, 12.7 mmol), and p-toluenesulfonic acid monohydrate (0.193 g, 1.02 mmol) in water (6 mL) was heated at 100° C. for 24 hours. KOH (1.78 g, 31.7 mmol) in water (4 mL) is added and the mixture is cooled to 50° C., treated with decolorizing charcoal, and filtered. The pH is adjusted to 5 with sulfuric acid resulting in precipitation of a white solid. The precipitate was recrystallized in methanol, washed with water, and dried under vacuum. NMR of the recrystallized solid matched the literature values for the title compound, 1.01 (2.40 g, 72.1%).

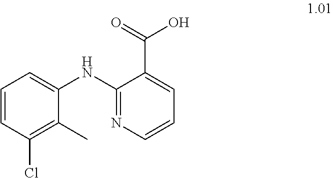 [SMILES: Cc1c(Cl)cccc1Nc1ncccc1C(=O)O]

To a solution of 1.01 (0.65 g 2.47 mmol) in anhydrous DCM (18 mL) was added oxalyl chloride (0.260 mL, 2.97 mmol) and anhydrous DMF (3-5 drops) under argon atmosphere. After 1 hour, the reaction mixture was concentrated in vacuo to dryness. N, O dimethylhydroxylamine hydrochloride (0.362 g, 3.71 mmol) was suspended anhydrous DCM (18 mL) and anhydrous TEA (0.862 mL, 6.19 mmol) and cooled to 0° C. under argon atmosphere. The newly formed acid chloride was in DCM (18 mL) and slowly added to the cooled solution under argon atmosphere. The mixture was then warmed to rt, stirred for 4 hours, then washed with saturated NH4Cl solution, water, and dried over MgSO₄. The crude reaction mixture was passed through a silica plug (1:4 diethyl ether: DCM) and concentrated under vacuum to give 1.06 as a white solid. This compound was brought to the next step without further purification.

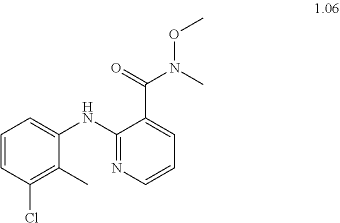 [SMILES: CON(C)C(=O)c1cccnc1Nc1cccc(Cl)c1C]

In a round bottom flask, 1.06 (0.150 g, 0.491 mmol) was dissolved in THF (2 mL) and brought to −78° C. while stirring, followed by the addition of n-BuLi (0.307 ml, 0.491 mmol) was added dropwise. The reaction was left for 1 hour brought to room temperature. The reaction was quenched with NH4Cl and extracted with ethyl acetate. The crude organic material was purified via column chromatography (1:4 diethyl ether: DCM) to yield 1.07 (0.084 g, 56.5%). HRMS m/z: [M+H]⁺calc'd for C₁₇H₂₀CIN₂O 303.1259; Found 303.1269 ppm.

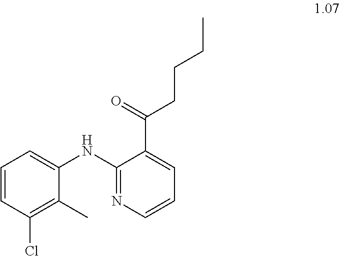 [SMILES: CCCCC(=O)c1cccnc1Nc1cccc(Cl)c1C]

Disclosed herein are compounds having a structure:

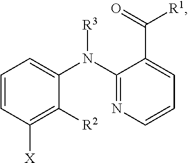

wherein R¹ is a C1-C12 branched or unbranched alkyl or branched or unbranched cycloalkyl group which is substituted with 0, 1, 2, 3, or 4 groups selected from halogen,—CN,—NH₂,—OH, and —NO₂; wherein R² is a C1-C4 branched or unbranched alkyl or branched or unbranched cycloalkyl group which is substituted with 0, 1, or 2 groups selected from halogen, —CN, —NH₂, —OH, and —NO₂; wherein R³ is —H, —CH₃, or —CH₂CH₃; and wherein X is —F, —Cl, —Br, or —I. In some aspects, the compound has a structure selected from:

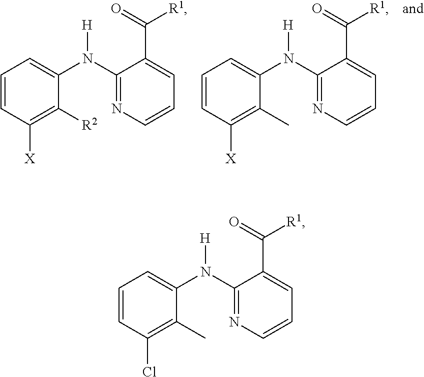

wherein R¹ is a C1-C12 branched or unbranched alkyl or branched or unbranched cycloalkyl group which is substituted with 0, 1, 2, 3, or 4 groups selected from halogen, —CN, —NH₂, —OH, and —NO₂; wherein R² ; is a C1-C4 branched or unbranched alkyl or branched or unbranched cycloalkyl group which is substituted with 0, 1, or 2 groups selected from halogen, —CN, —NH₂, —OH, and —NO₂; and wherein X is —F, —Cl, —Br, or —I. In some aspects, the compound has a

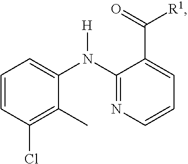

wherein R¹ is a C1-C12 branched or unbranched alkyl or branched or unbranched cycloalkyl group which is substituted with 0, 1, or 2 groups selected from halogen, —CN, —NH₂, —OH, and —NO₂. In some aspects, wherein R¹ can be a C1-C6 branched or unbranched alkyl group. In some aspects, the compound is not Clonixin Butyl Ketone.

In some aspects, the compounds, compositions, or pharmaceutical compositions disclosed herein can be in an aqueous form.

### Pharmaceutical Compositions

Disclosed herein are pharmaceutical composition comprising a pharmaceutically acceptable carrier and an active ingredient selected from Clonixeril and a Clonixin Ketone Analog. In some aspects, the active ingredient can be a Clonixin Ketone Analog having a structure:

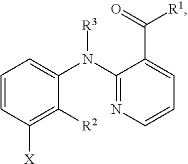

wherein R¹ is a C1-C12 branched or unbranched alkyl or branched or unbranched cycloalkyl group which is substituted with 0, 1, 2, 3, or 4 groups selected from halogen, —CN, —NH₂, —OH, and —NO₂; wherein R² is a C1-C4 branched or unbranched alkyl or branched or unbranched cycloalkyl group which is substituted with 0, 1, or 2 groups selected from halogen, —CN, —NH₂, —OH, and —NO₂; wherein R³ is —H, —CH₃, or —CH₂CH₃; and wherein X is —F, —Cl, —Br, or —I. In some aspects, the Clonixin Ketone Analog has a structure selected from:

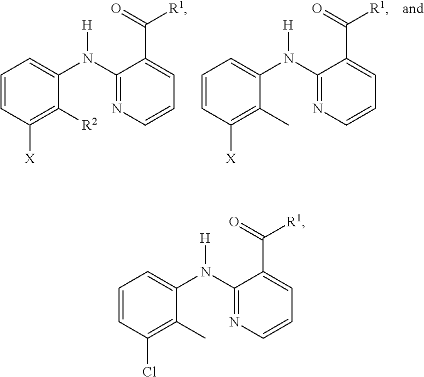

wherein R¹ is a C1-C12 branched or unbranched alkyl or branched or unbranched cycloalkyl group which is substituted with 0, 1, 2, 3, or 4 groups selected from halogen, —CN, —NH₂,—OH, and —NO₂; wherein R² is a C1-C4 branched or unbranched alkyl or branched or unbranched cycloalkyl group which is substituted with 0, 1, or 2 groups selected from halogen, —CN, —NH₂, —OH, and —NO₂; and wherein X is —F, —Cl, —Br, or —I. In some aspects, the compound has a structure:

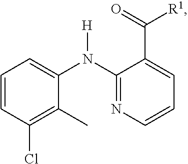

wherein R^{1 is a C}1-C12 branched or unbranched alkyl or branched or unbranched cycloalkyl group which is substituted with 0, 1, or 2 groups selected from halogen, —CN, —NH₂, —OH, and —NO₂. In some aspects, R¹ can be a C1-C6 branched or unbranched alkyl group. In some aspects, the compound is not Clonixin Butyl Ketone.

As disclosed herein, are pharmaceutical compositions, comprising one or more of the therapeutic compositions or STING inhibitors disclosed herein. In some aspects, the compositions can be formulated for oral or parental administration. In some aspects, the parental administration can be intravenous, subcutaneous, intramuscular, or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions are administered to a subject (e.g., a human patient) already with or diagnosed with a traumatic brain injury in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the traumatic brain injury is delayed, hindered, or prevented, or the traumatic brain injury or a symptom of the traumatic brain injury is ameliorated. One or more of the symptoms can be less severe. Recovery can be accelerated in an individual who has been treated.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of traumatic brain injury.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, acrosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. In some aspects, the composition can be prepared for intravenous, subcutaneous, intramuscular, oral, or intranasal administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes H151, clonixeril, a ketone analogue of clonixeril, clonixin, a ketone analogue of clonixin, or a STING inhibitor dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

In some aspects, the formulations disclosed herein can vary or be tailored according to the disease, disorder, or condition or the severity of the disease, disorder, or condition to be treated, the amount of compound to be administered, the condition of the individual, and other variables that will readily be apparent to one of ordinary skill in the art in view of the teachings provided herein. In some aspects, the compositions disclosed herein can be formulated for delivery to the nasal cavity. In some aspects, the compositions disclosed herein can be formulated as a powered spray, a liquid spray, nose drops, a gel, or an ointment.

### Methods of Treatment

Disclosed herein are methods of treating a subject with a traumatic brain injury. In some aspects, the methods can comprise administering to the subject a composition comprising a therapeutically effective amount of H151, clonixeril, a ketone analogue of clonixeril, clonixin, a ketone analogue of clonixin, a STING inhibitor, or a combination thereof, thereby treating the subject with the traumatic brain injury. In some aspects, the traumatic brain injury can be caused by a head injury. In some aspects, the composition can by administered intravenously, subcutaneously, intramuscularly, orally, or intranasally.

Disclosed herein are methods of reducing brain inflammation in a subject with a traumatic brain injury. In some aspects, the methods can comprise administering to the subject a composition comprising a therapeutically effective amount of H151, clonixeril, a ketone analogue of clonixeril, clonixin, a ketone analogue of clonixin, a STING inhibitor, or a combination thereof, thereby reducing brain inflammation the subject with the traumatic brain injury. In some aspects, the traumatic brain injury can be caused by a head injury. In some aspects the brain inflammation can be caused by primary or secondary traumatic brain injury due to a head injury. In some aspects, the composition can by administered intravenously, subcutaneously, intramuscularly, orally, or intranasally.

Disclosed herein are methods of reducing or inhibiting IBA1 or pSTING. In some aspects, the methods can comprise administering to a subject a composition comprising a therapeutically effective amount of H151, clonixeril, a ketone analogue of clonixeril, clonixin, a ketone analogue of clonixin, a STING inhibitor, or a combination thereof, thereby reducing or inhibiting IBA1 or pSTING. In some aspects, the subject has a traumatic brain injury. In some aspects, the traumatic brain injury can be caused by a head injury. In some aspects, the composition can by administered intravenously, subcutaneously, intramuscularly, orally, or intranasally.

Disclosed herein are method of reducing immune markers of inflammation. In some aspects, the immune markers of inflammation can be one or more of IRF3, IFNβ, CCL20, CCR6 and IL-1⊕. Also disclosed herein are methods of reducing expression of IRF3, IFNβ, CCL20, CCR6 or IL-1β in a subject. In some aspects, the methods can comprise administering to the subject a composition comprising a therapeutically effective amount of H151, clonixeril, a ketone analogue of clonixeril, clonixin, a ketone analogue of clonixin, a STING inhibitor, or a combination thereof, thereby reducing expression of IRF3, IFNβ, CCL20, CCR6 or IL-1β in the subject. In some aspects, the subject has a traumatic brain injury. In some aspects, the traumatic brain injury can be caused by a head injury. In some aspects, the composition can by administered intravenously, subcutaneously, intramuscularly, orally, or intranasally. In some aspects, the method can comprise administering to the subject a therapeutically effective amount of clonixeril, a ketone analogue of clonixeril, clonixin, a ketone analogue of clonixin, thereby reducing expression of CCL20 and IL-1β. In some aspects, the method can comprise administering to the subject a therapeutically effective amount of H151, thereby reducing expression of CCL20 and IL-1β.

Disclosed herein are methods of reducing neuroinflammation in a subject. In some aspects, the methods of reducing neuroinflammation can be measured by expression of IBA1 in the subject. Also disclosed herein are methods of reducing expression of ionized calcium-binding adaptor molecule 1 (IBA1) in a subject. In some aspects, the methods can comprising administering to the subject a composition comprising a therapeutically effective amount of H151, clonixeril, a ketone analogue of clonixeril, clonixin, a ketone analogue of clonixin, a STING inhibitor, or a combination thereof, thereby reducing expression IBA1 the subject. In some aspects, the subject has a traumatic brain injury. In some aspects, the traumatic brain injury can be caused by a head injury. In some aspects, the composition can by administered intravenously, subcutaneously, intramuscularly, orally, or intranasally.

In some aspects, the subject has a traumatic brain injury. In some aspects, the traumatic brain injury can be caused by a head injury. In some aspects, the head injury can be caused be a fall. In some aspects, the traumatic brain injury can be caused by a concussion. In some aspects, the traumatic brain injury can be the result when the head suddenly and violently hits an object or when an object pierces the skull and enters brain tissue.

In some aspects, the subject has been diagnosed with a traumatic brain injury prior to the administering step.

In some aspects, a subject “in need thereof” can be an individual who has been diagnosed with, previously treated for, and/or suspected of having the disease or condition to be treated. With respect to prevention, the individual in need thereof may also be an individual who is at risk for a disease or condition (e.g., occupation (construction workers, profession drivers, emergency responders, military service, etc.).

In some aspects, the disease or condition can be accompanied by one or more symptoms of TBI. In some aspects, the disease or condition can be accompanied by inflammation (e.g., neuroinflammation). In some aspects, the acetaminophen or analog thereof and/or formulation comprising the H151, clonixeril, a ketone analogue of clonixeril, clonixin, a ketone analogue of clonixin, or a STING inhibitor can reduce the severity of one or more symptoms associated with a disease or condition that is responsive to H151, clonixeril, a ketone analogue of clonixeril, clonixin, a ketone analogue of clonixin, or a STING inhibitor by at least about any of 10%, 20%, 30%, 40%, 50%, 60%, 70%, 80%, 90%, 95%, or 100% compared to the corresponding symptom in the same subject prior to treatment or compared to the corresponding symptom in other subjects not receiving the H151, clonixeril, a ketone analogue of clonixeril, clonixin, a ketone analogue of clonixin, or a STING inhibitor.

In some aspects, the administration of any of compositions described herein can reduce one or more of the symptoms of a traumatic brain injury. Examples of one or more symptoms of TBI include but are not limited to physical symptoms, sensory symptoms, and cognitive, behavior, or mental symptoms. Examples of physical symptoms of TBI include but are not limited to headache, nausea or vomiting, fatigue or drowsiness, problems with speech, dizziness or loss of balance, loss of consciousness, convulsions or seizures, dilation of one or both pupils of the eyes, clear fluids draining from the nose or ears, inability to awaken from sleep, weakness of numbness in fingers and toes, and loss of coordination. Examples of sensory symptoms of TBI include but are not limited to blurred vision, ringing in the cars, a bad taste in the mouth, or changes in the ability to smell. Examples of cognitive, behavioral or mental symptoms of TBI include but are not limited to loss of consciousness, a state of being dazed, confused (or profound confusion), or disoriented, memory or concentration problems, mood changes or mood swings, feeling depressed or anxious, difficulty sleeping, sleeping more than usual, agitation, combativeness or other unusual behavior, slurred speech, or coma and other disorders of consciousness.

In any of the methods disclosed herein, the ketone analogue of clonixin can be clonixin butyl ketone.

In any of the methods disclosed herein, the ketone analogue of clonixin can a compound having a structure:

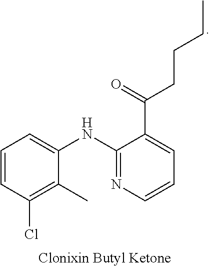 [SMILES: CCCCC(=O)c1cccnc1Nc1cccc(Cl)c1C]

In any of the methods disclosed herein, wherein the ketone analogue of clonixin comprises a compound having a structure:

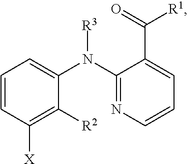

wherein R¹ is a C1-C12 branched or unbranched alkyl or branched or unbranched cycloalkyl group which is substituted with 0, 1, 2, 3, or 4 groups selected from halogen, —CN, —NH₂, —OH, and —NO₂;

wherein R² is a C1-C4 branched or unbranched alkyl or branched or unbranched cycloalkyl group which is substituted with 0, 1, or 2 groups selected from halogen, —CN, —NH₂, —OH, and —NO₂; wherein R³ is —H, —CH₃, or —CH₂CH₃; and wherein X is —F, —Cl, —Br, or —I.

The therapeutically effective amount or dosage of the H151, clonixeril, a ketone analogue of clonixeril, clonixin, a ketone analogue of clonixin, or a STING inhibitor used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the TBI symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. The present compounds can be given to prior to, simultaneously with or after treatment with other agents or regimes. For example, H151, clonixeril, a ketone analogue of clonixeril, clonixin, a ketone analogue of clonixin, or a STING inhibitor disclosed herein can be administered in conjunction with standard therapies used to treat TBI (or inflammation).

### Articles of Manufacture

The compositions and pharmaceutical compositions described herein can be packaged in a suitable container labeled, for example, for use as a therapy to treat TBI or any of the methods disclosed herein. Accordingly, packaged products (e.g., sterile containers containing the composition described herein and packaged for storage, shipment, or sale at concentrated or ready-to-use concentrations) and kits, including at least H151, clonixeril, a ketone analogue of clonixeril, clonixin, a ketone analogue of clonixin, or a STING inhibitor as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio-or videotape)). The legend can be associated with the container (e.g., affixed to the container) and can describe the manner in which the compound therein should be administered (e.g., the frequency and route of administration), indications therefor, and other uses. The compositions can be ready for administration (e.g., present in dose-appropriate units), and may include a pharmaceutically acceptable adjuvant, carrier or other diluent. Alternatively, the compounds can be provided in a concentrated form with a diluent and instructions for dilution.

### EXAMPLES

### Example 1: Therapeutic Potential of STING Inhibition in Traumatic Brain Injury

FIG. 1 shows that TBI induces immune response protein expression in the hippocampus. IPA proteomics pathway analysis shows multiple immune response proteins are significantly increased following TBI, and that these increases directly lead to increased STING expression. Stopping STING reduced the levels of several important factors, including IRF3, IFNβ, CCL20, and CCR6, which are linked to brain damage and inflammation (FIG. 1).

These findings demonstrate that targeting STING can reduce brain inflammation and improve recovery from TBI (FIG. 2).

### Example 2: Inhibition of STING Reduces Microglial Activation for Rescue of Traumatic Brain Injury

Experimental design: IMG or BV2 cells were plated in a 96-well or 6-well plate and allowed to adhere overnight. Plates were dosed with clonixeril for 2 hours and then LPS or polyinosinic-polycytidylic acid (polyIC) for 24 hours to induce inflammation. Ionized calcium-binding adaptor molecule 1 (IBA1) and pSTING expression were imaged and immunohistochemistry was measured (ImageJ).

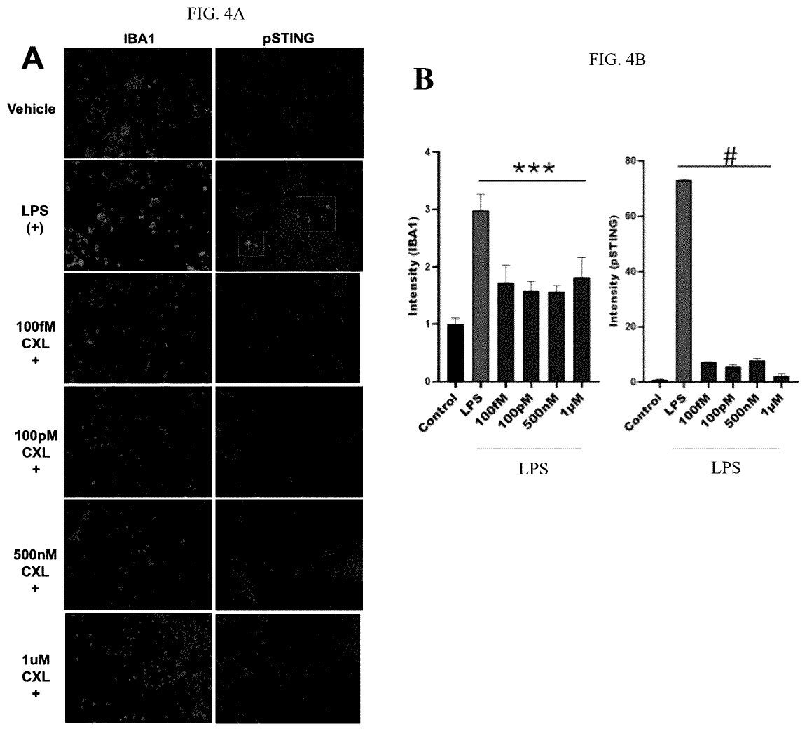

FIG. 4A shows immunocytochemical analysis demonstrating a reduction in IBA1 (a marker for microglial activation) and pSTING in LPS-stimulated immature microglial cells (IMG). Cells were untreated, or pre-treated with vehicle or STING-inhibitory compound clonixeril for 2 h and then treated with LPS for 24 h. Cells were then stained with DAPI (blue) to visualize nuclei, IBA1 or pSTING antibody (red). Images are presented separately for enhanced clarity. FIG. 4B shows that quantification of integrated density in LPS-stimulated IMG cells demonstrate a reduction in IBA1 and pSTING following pre-treatment with STING-inhibitory compound, clonixeril, and treatment with LPS for 24 h. Error bars represent mean ±standard deviation (SD). Separate channels were quantified by ImageJ for IBA1 or pSTING (red) and DAPI (blue) pixel density. A ratio of red signal to blue signal was calculated for each image. Ratios were averaged for each experimental group and normalized to the blank (control) group.

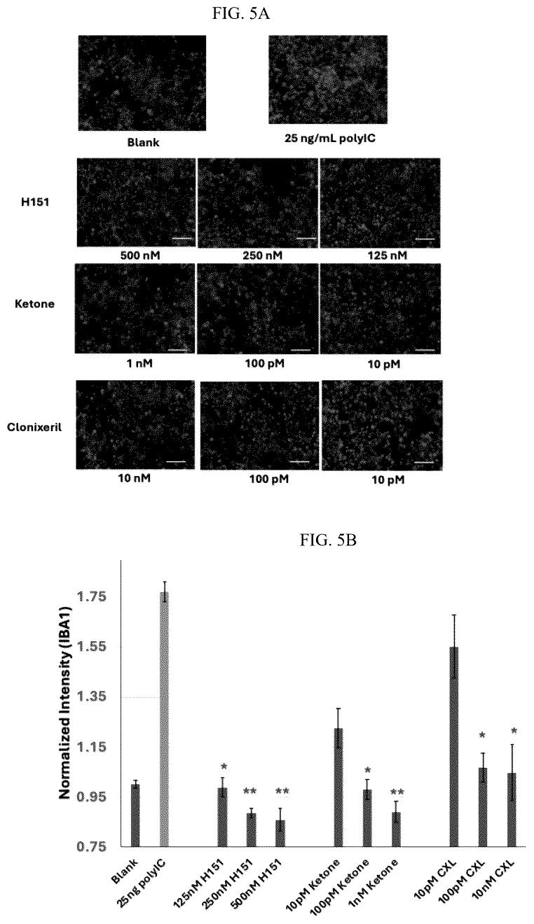

FIG. 5A shows immunocytochemical analysis demonstrating a reduction in IBA1 of poly (I:C)-stimulated BV2 microglial cells treated with STING-inhibitory compounds H151, clonixeril or a ketone analogue of clonixin (e.g., clonixin butyl ketone). Controls used are a blank (untreated); and 25 ng/ml poly (I:C) treatment for 6 h. Immunocytochemistry of BV2 cells pre-treated with a known STING inhibitor H151, clonixeril or a ketone analogue of clonixin (e.g., clonixin butyl ketone) for 2 h and then treated with poly (I:C) for 6 h, stained with IBA1 antibody (red) and DAPI (blue) to visualize nuclei. FIG. 5B shows that the quantification of integrated density in poly (I C)-stimulated BV2 microglial cells demonstrates a reduction in IBA1 following treatment with STING-inhibitory compounds. Separate channels were quantified by ImageJ for IBA1 (red) and DAPI (blue) pixel density. A ratio of red signal to blue signal was calculated for each image. Ratios were averaged for each experimental group and normalized to the blank (control) group.

The results show that blocking the STING pathway reduces brain inflammation and improves recovery after traumatic brain injury (TBI). More specifically, lab studies on microglial cells (e.g., IMG and BV2) showed that blocking the STING pathway reduces the activity of harmful proteins and genes that contribute to inflammation after a traumatic brain injury (TBI).

### Example 3: Inhibition of STING Reduces Expression of TBI-Related Inflammatory and Immunomodulatory Genes

These results show that blocking the STING pathway can help improve recovery from TBI by reducing harmful proteins (e.g., CCL20 and CCR6) that cause brain inflammation. Studies on mice with brain injuries showed that when STING is activated, it leads to inflammation and damage to brain cells. Further lab tests on brain cells showed that stopping STING helped reduce important signs of inflammation. In sum, these findings demonstrate that targeting STING can be a helpful treatment for TBI recovery.

This study aims to reduce the activation of CCL20 and CCR6 through drug candidate inhibition of STING, which is activated by TBI and acts as a direct promotor to these chemokines. This work will directly aid in the rehabilitation of U.S. service members, significantly improve their overall quality of life, and reduce costs associated with long term disability. FIG. 2 shows how TBI is affected by STING.

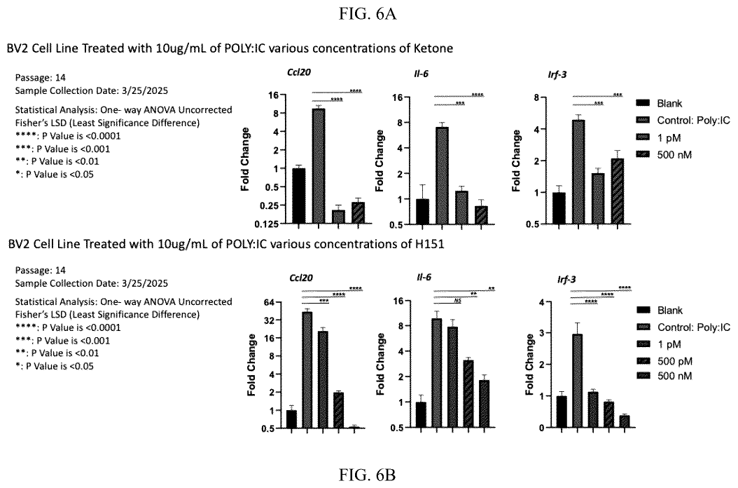

Experimental design: BV2 cells were plated in a 6-well plate and allowed to adhere overnight. Plates were dosed with clonixin butyl ketone or H151 for 2 hours and then PolyIC for 24 hours to induce inflammation. qPCR was performed to measure expression of CCL20 and IL-6 inflammatory genes and immunomodulatory Irf-3 gene. FIG. 6 shows that treatment with clonixin butyl ketone or H151 significantly decreased expression of inflammatory markers, such as CCL20 and IL-6. It also reduced significantly interferon regulatory gene, Irf3. Importantly, clonixin butyl ketone but not H151 showed a significant reduction of CCL20 and IL-6 expression at 1 picomolar concentration.

### Example 4: Inhibition of pSTING in Hippocampal Neuronal Cells

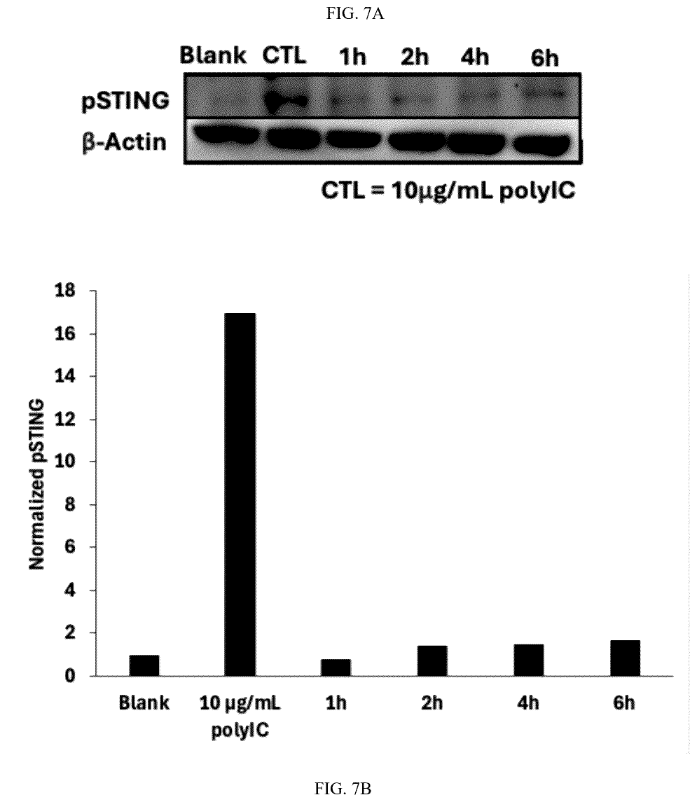

FIG. 7. shows inhibition of poly (I:C)-induced STING phosphorylation by a ketone analog of clonixeril (e.g., clonixin butyl ketone) in HT22 hippocampal cells by Western blot. Immunoblot analysis of phosphorylated STING (pSTING) and β-actin in HT22 mouse hippocampal cells. Cells were pre-treated with 100 pM ketone analog of clonixin (e.g., clonixin butyl ketone) for 2 h, followed by 6 h stimulation with poly (I:C). Cell lysates were harvested at indicated time points using RIPA buffer supplemented with 1× Halt Protease and Phosphatase Inhibitor Cocktail, quantified via Bradford assay, and normalized to 20 μg protein per well. Lysates were resolved by SDS-PAGE and probed with rabbit polyclonal anti-pSTING and anti-β-actin antibodies. FIG. 7A shows Western blot for anti-pSTING (top) and anti-β-actin (bottom). β-actin served as the loading control. FIG. 7B shows the quantification of pSTING expression, normalized to β-actin, using ImageJ analysis of band densitometry.

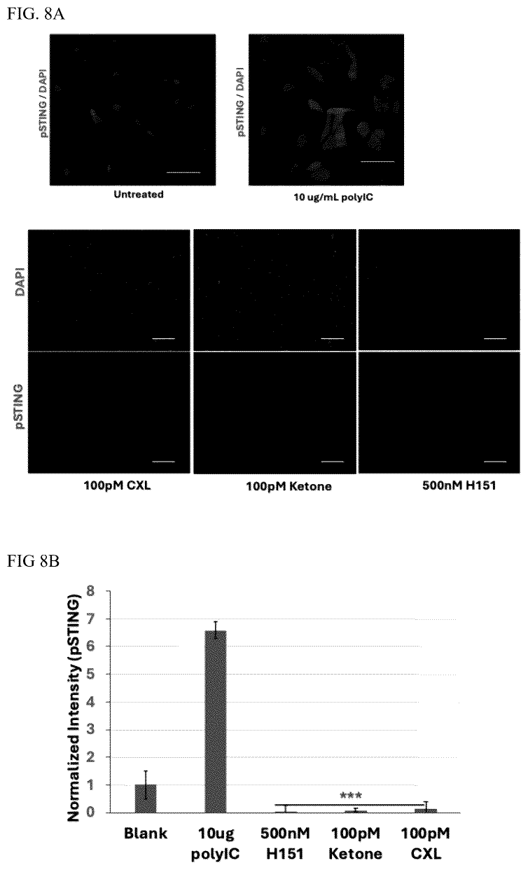

FIG. 8A shows inhibition of poly (I:C)-induced STING phosphorylation by clonixeril (CXL), ketone analog of clonixin (e.g., clonixin butyl ketone) or H151 in HT22 hippocampal cells by immunohistochemistry. Immunocytochemical analysis demonstrates a reduction in pSTING of poly (I:C)-stimulated HT22 mouse hippocampal cells. Cells were untreated, or pre-treated with vehicle or STING-inhibitory compounds H151, a ketone analog of clonixin (e.g., clonixin butyl ketone), clonixeril (CXL) for 2 h and then treated with poly (I:C) for 6 h. Cells were then stained with DAPI (blue, top row) to visualize nuclei and pSTING antibody (red, bottom 5 row). Images are presented separately for enhanced clarity. Scale bar 100 μm.

FIG. 8B shows that quantification of integrated density in poly (I:C)-stimulated HT22 mouse hippocampal cells demonstrate a reduction in pSTING following pre-treatment with STING-inhibitory compounds and treatment with poly (I:C) for 6 h. Error bars represent mean ±standard deviation (SD). Separate channels were quantified by ImageJ for pSTING (red) and DAPI (blue) pixel density. A ratio of red signal to blue signal was calculated for each image. Ratios were averaged for each experimental group and normalized to the blank (control) group.

1.-6. (canceled)

7. A method of reducing or inhibiting IBA1 or pSTING, the method comprising administering to a subject a composition comprising a therapeutically effective amount of H151, clonixeril, a ketone analogue of clonixeril, clonixin, a ketone analogue of clonixin, or a combination thereof, thereby reducing or inhibiting IBA1 or pSTING.

8. The method of claim 7, wherein the subject has a traumatic brain injury.

9. The method of claim 7, wherein the composition is administered intravenously, subcutaneously, intramuscularly, orally, or intranasally.

10.-16. (canceled)

17. A method of treating a subject with a traumatic brain injury, the method comprising administering a therapeutically effective amount of a compound having a structure: 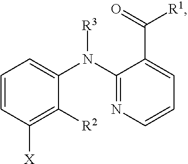
wherein R¹ is a C1-C12 branched or unbranched alkyl or branched or unbranched cycloalkyl group which is substituted with 0, 1, 2, 3, or 4 groups selected from halogen, —CN, —NH₂, —OH, and —NO₂;
wherein R² is a C1-C4 branched or unbranched alkyl or branched or unbranched cycloalkyl group which is substituted with 0, 1, or 2 groups selected from halogen, —CN, —NH₂, —OH, and —NO₂;
wherein R³ is —H, —CH₃, or —CH₂CH₃; and
wherein X is —F, —Cl, —Br, or —I.

18. The method of claim 17, wherein the compound has a structure selected from: 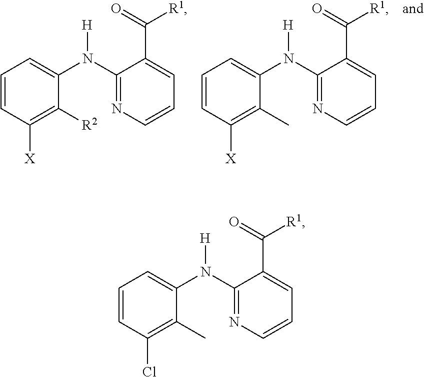
wherein R¹ is a C1-C12 branched or unbranched alkyl or branched or unbranched cycloalkyl group which is substituted with 0, 1, 2, 3, or 4 groups selected from halogen, —CN, —NH₂, —OH, and —NO₂;
wherein R² is a C1-C4 branched or unbranched alkyl or branched or unbranched cycloalkyl group which is substituted with 0, 1, or 2 groups selected from halogen, —CN, —NH₂, —OH, and —NO₂; and
wherein X is —F, —Cl, —Br, or —I.

19. The method of claim 17, wherein the compound has a structure: 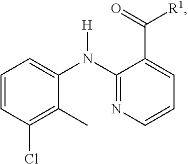
wherein R¹ is a C1-C12 branched or unbranched alkyl or branched or unbranched cycloalkyl group which is substituted with 0, 1, or 2 groups selected from halogen, —CN, —NH₂, —OH, and —NO₂.

20. The method of claim 19, wherein R¹ is a C1-C6 branched or unbranched alkyl group.

21. A compound having a structure: 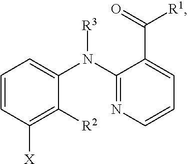
wherein R¹ is a C1-C12 branched or unbranched alkyl or branched or unbranched cycloalkyl group which is substituted with 0, 1, 2, 3, or 4 groups selected from halogen, —CN, —NH₂, —OH, and —NO₂;
wherein R² is a C1-C4 branched or unbranched alkyl or branched or unbranched cycloalkyl group which is substituted with 0, 1, or 2 groups selected from halogen, —CN, —NH₂, —OH, and —NO₂;
wherein R³ is —H, —CH₃, or —CH₂CH₃; and
wherein X is —F, —Cl, —Br, or —I.

22. The compound of claim 21, wherein the compound has a structure selected from: 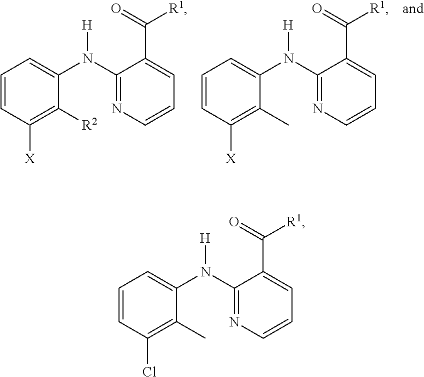
wherein R¹ is a C1-C12 branched or unbranched alkyl or branched or unbranched cycloalkyl group which is substituted with 0, 1, 2, 3, or 4 groups selected from halogen, —CN, —NH₂, —OH, and —NO₂;
wherein R² is a C1-C4 branched or unbranched alkyl or branched or unbranched cycloalkyl group which is substituted with 0, 1, or 2 groups selected from halogen, —CN, —NH₂, —OH, and —NO₂; and
wherein X is —F, —Cl, —Br, or —I.

23. The compound of claim 21, wherein the compound has a structure: 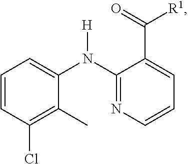
wherein R¹ is a C1-C12 branched or unbranched alkyl or branched or unbranched cycloalkyl group which is substituted with 0, 1, or 2 groups selected from halogen, —CN, —NH₂, —OH, and —NO₂.

24. The compound of claim 23, wherein R¹ is a C1-C6 branched or unbranched alkyl group.

25. The compound of claim 21, wherein the compound is not Clonixin Butyl Ketone.

26. A pharmaceutical composition comprising the compound of claim 21 and a pharmaceutically acceptable carrier.

27.-31. (canceled)

32. The method of claim 7, wherein the ketone analogue of clonixin is clonixin butyl ketone.

33. The method of claim 7, wherein the ketone analogue of clonixin is a compound having a structure:

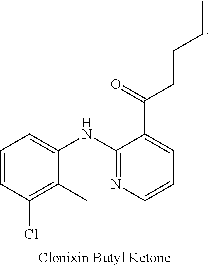 [SMILES: CCCCC(=O)c1cccnc1Nc1cccc(Cl)c1C]

34. The method of claim 7, wherein the ketone analogue of clonixin comprises a compound having a structure: 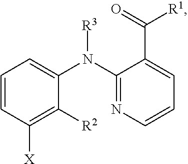
wherein R¹ is a C1-C12 branched or unbranched alkyl or branched or unbranched cycloalkyl group which is substituted with 0, 1, 2, 3, or 4 groups selected from halogen, —CN, —NH₂, —OH, and —NO₂;
wherein R² is a C1-C4 branched or unbranched alkyl or branched or unbranched cycloalkyl group which is substituted with 0, 1, or 2 groups selected from halogen, —CN, —NH₂, —OH, and —NO₂;
wherein R³ is —H, —CH₃, or —CH₂CH₃; and
wherein X is —F, —Cl, —Br, or —I.

35. A method of reducing expression of IRF3, IFNβ, CCL20, CCR6 or IL-1β in a subject, the method comprising administering to the subject a therapeutically effective amount of the compound of claim 21, thereby reducing expression of IRF3, IFNβ, CCL20, CCR6 or IL-1β in the subject.

36. A method of reducing brain inflammation in a subject with a traumatic brain injury, the method comprising administering to the subject a therapeutically effective amount of the compound of claim 21, thereby reducing brain inflammation the subject with the traumatic brain injury.
